(19) United States
(12) Patent Application Publication
(10) Pub. No.: US 2008/0130830 A1
(43) Pub. Date: Jun. 5, 2008

(54) Radiation Image Capturing System for Mammography and Radiation Image Conversion Panel

(76) Inventor: Tomoko Saito, Tokyo (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/795,481

(22) PCT Filed: Jan. 17, 2006
(86) PCT No.: PCT/JP06/00522

§ 371 Date: Jul. 17, 2007

(30) Foreign Application Priority Data
Jan. 26, 2005 (JP) 2005-017887

(51) Int. Cl. A61B 6/00

(52) U.S. Cl. 378/37

(57) ABSTRACT

A radiation image capturing system for mammography comprising: a radiation generator exposing a subject to a radiation; an image capturing stage; a compressing plate compressing the subject; and a radiation image conversion panel recording image information of radiation transmitted through the subject, wherein, emission luminance of the radiation image conversion panel on a side opposite to a chest wall side is higher than luminance on the chest wall side.

### TECHNICAL FIELD

The present invention relates to a radiation image capturing system for mammography and a radiation image conversion panel used for the same.

### BACKGROUND OF THE INVENTION

Heretofore, radiation images typically X-ray images have been widely employed for medical diagnosis.

In recent years, instead of radiographic systems which employ an intensifying screen and silver halide photographic materials, practiced have been radiation image recording and reproducing systems employing “stimulable phosphors” which accumulate exposed radiation energy and result in photostimulated luminescence depending on accumulated radiation energy when exposed to exciting radiation.

In the above systems, by exposing radiation which has passed through a subject to stimulable phosphors, radiation energy (hereinafter referred to as “image information”) corresponding to radiation transmission density of each portion of the subject is accumulated in stimulable phosphors. Thereafter, image information accumulated in stimulable phosphors is released as photostimulated luminescence, employing exciting radiation, and variation of the resulting photostimulated luminescence is converted to electric signals, whereby the image information is reproduced as visible images, employing image recording materials such as photosensitive materials or image display devices such as CRT.

In many cases, sheet-like stimulable phosphors (hereinafter referred to as stimulable phosphor sheets), which are employed in the above radiation image recording system, are arranged in an enclosure while fixed on a specified supporting plate and are used for radiation image capturing. After capturing of radiation images, image information stored in the stimulable phosphors are read via a radiation image reading means (hereinafter referred to as “reading means”).

Mammography (X-ray breast imaging) is one of the unavoidable examination methods for identification diagnosis of breast diseases, particularly breast cancer. Mammography exhibits the following advantages. It is possible to discover minute breast cancer via mammography examination for superficially healthy breasts without a “lump”.

In advanced countries such as European countries and The United States, where one of twenty females contracts breast cancer, mammography has previously been employed during breast cancer examinations. On the other hand, the breast cancer rate in our country is still low compared to European countries and the United States. However, along with our westernization of everyday living, breast cancer has rapidly increased. Consequently, in our country, a system to introduce mammography for breast cancer examination is to be shortly established.

In practical image capturing, it is common that in the X-ray exposure dose distribution in the radiation image capturing system (being the apparatus), X-ray exposure dose decreases from the chest wall side to the non-chest wall side.

On the other hand, any of the common radiation image recording media such as S/F, CR, or FPD exhibit a linear correlation between X-ray dose and emission luminance (namely output intensity of the images). Further, pursued heretofore have been radiation image conversion panels which precisely correspond to X-rays at a specified definite amount and result in uniform emission luminance (refer, for example, to Patent Document 1). Accordingly, as noted above, radiation images formed by exposure to X-rays having a distribution from an image capturing apparatus tend to have a luminance distribution (namely a density distribution) reflecting the above X-ray distribution.

In mammography radiation images, it is preferable that images at relatively high density not to interrupt diagnosis are obtained in the peripheral breast region which is specifically important for diagnosis. However, at present, image density does not satisfy the values suitable for precise diagnosis on the non-breast wall side of the panel because of the abovementioned distribution of X-ray dose caused by the apparatus.

As a means to overcome the above drawbacks, it is proposed to perform image processing which increases output image density in the peripheral breast portions (refer to, for example, to Patent Document 2). However, there have been problems that it takes time to recognize the breast outline or the accuracy in recognizing the breast outline tends to fluctuate, in the image processing.

(Patent Document 1) Japanese Patent Application Publication Open to Public Inspection (hereinafter referred to as JP-A) No. 2002-250797

(Patent Document 2) JP-A No. 2000-163562

### SUMMARY OF THE INVENTION

In view of the above drawbacks, the present invention was achieved. An object of the present invention is to provide a radiation image capturing system for mammography which results in high density images suitable for diagnosis, specifically in the peripheral breast portions, which are specifically required for diagnosis, even though X-ray dose decreases from the near chest wall side to the side opposite to chest wall side (hereafter also referred to as the non-chest wall side), and to provide a radiation image conversion panel which is suitably used in the application of the same.

One of the embodiments to achieve the above object is a radiation image capturing system for mammography comprising the steps of: exposing a subject to a radiation; and recording image information of radiation transmitted through the subject on a radiation image conversion panel, wherein, emission luminance of the radiation image conversion panel on a side opposite to a chest wall side is higher than luminance on the chest wall side.

### BRIEF DESCRIPTION OF THE DRAWINGS

FIG. 1 is a conceptual view of the radiation image conversion method employed in a radiation image detecting medium (being a radiation image conversion panel).

FIG. 2 is a schematic view of the structure of a radiation image capturing system for mammography.

FIG. 3 is distribution of X-ray intensity (X-ray dose) which reaches a subject.

FIG. 4 is a schematic view of a state in which X-rays having a distribution of X-ray dose is exposed to a subject so that an image is captured.

FIG. 5(a) is a sectional view of an outlet for coating liquid of a slide hopper, in which the size of the aperture varies in the width direction.

FIG. 5(b) is a sectional view of an outlet for coating liquid of a slide hopper, in which the size of the aperture is uniform in the width direction.

FIG. 6(a) is a sectional view of a stimulable phosphor layer applied onto a support in such a manner that the thickness varies in the width direction.

FIG. 6(b) is a sectional view of a stimulable phosphor layer applied onto a support in such a way that the thickness is uniform in the width direction.

FIG. 7 is a view explaining a method which gradually increases the surface area of a evaporation source to change the deposited amount onto a support.

FIG. 8 is a view explaining a coating method in which the outlet for coating liquid of a slide hopper is divided into several portions, and stimulable phosphor coating liquids giving different emission luminances in the width direction are applied onto a support.

### DESCRIPTION OF THE PREFERRED EMBODIMENTS

The above object of the present invention is achieved employing the following embodiments.

(1) A radiation image capturing system for mammography comprising the steps of:

exposing a subject to a radiation; and

recording image information of the radiation transmitted through the subject on a radiation image conversion panel, wherein an emission luminance of the radiation image conversion panel on a side opposite to a chest wall side is higher than an emission luminance on the chest wall side.

(2) The radiation image conversion panel used in the radiation image capturing system for mammography of Item (1), the radiation image conversion panel having the emission luminance of the radiation image conversion panel on the side opposite to the chest wall side higher than the emission luminance on the chest wall side.

In formation of X-ray images, in an area exposed to X-rays at a smaller dose than the desired dose, it is possible to prepare clear images for easy diagnosis, only when the amount of stimulable phosphors is increased depending on the X-ray dose. Namely, in mammography radiation image capturing, only by allowing a higher emission luminance on the non-chest wall side of the radiation image conversion panel than the emission luminance on the chest wall side, it is possible to prepare X-ray images which exhibits nearly constant density in non-image portions, and enables overall precise diagnosis.

The present invention will now be further detailed.

Initially, explanation of overall radiation image formation will be provided.

At present, the representative method include, by using a stimulable phosphor as a radiation image detecting medium, exposing a subject to radiation to form an image of X-ray transmitted through the subject, reading the image, and then displaying the image. Based on the above method, a transmission image is formed via the following processes.

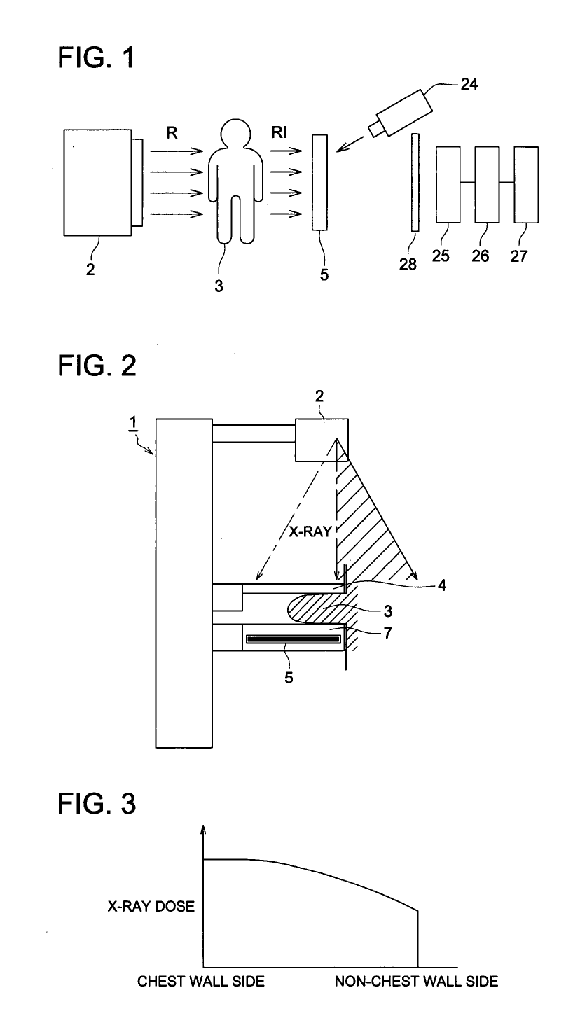

FIG. 1 conceptually shows a radiation image conversion method employing the radiation image detecting media (also referred to as radiation image conversion panels).

Namely, in FIG. 1, numeral 2 is a radiation generator, 3 is a subject (shown by a human body), 5 is a radiation image detecting medium according to the present invention, 24 is a stimulable excitation light source (such as a laser), 25 is a photoelectric conversion apparatus which detects stimulable fluorescence emitted from above radiation image detecting medium 5 via irradiation of stimulation excitation light, 26 is an apparatus which reproduces signals detected by 25 as an image, 27 is an apparatus which displays the reproduced image, while 28 is a filter which separates the stimulable excitation light and the stimulable fluorescence, and transmits only the stimulable fluorescence. Further, any of the members designated by the numbers of 25 or more are not specifically limited as long as they reproduce the light information from radiation image detecting medium 5 as an image.

As shown in FIG. 1, radiation (R) from radiation generator 2 is incident to radiation image detecting medium 5 through subject 3 (RI). The above incident radiation is absorbed by the stimulable layer of radiation image detecting medium 5 and its energy is accumulated, whereby an accumulated image of the transmitted radiation is formed.

Subsequently, the resulting accumulated image is excited by stimulable excitation light from stimulable excitation light source 24 to release photostimulated luminescence.

Since the intensity of the resulting photostimulated luminescence is proportional to the amount of the accumulated radiation energy, the image of radiation transmitted through a subject can be observed by photoelectrically convering the resulting radiation signals employing photoelectric conversion apparatus 25 such as a photoelectron multiplier tube, by reproducing an image employing image forming apparatus 26 and by displaying the image employing image display apparatus 27.

In the case of the radiation image capturing system for mammography, the tube voltage of radiation generator 2 is commonly lower than that for chest portion capturing and is commonly 20-35 kV.

### [Structure of Radiation Image Capturing System for Mammography of the Present Invention]

FIG. 2 shows the structure of the radiation image capturing system for mammography of the present invention.

Numeral 1 is a radiation image capturing apparatus for mammography. Subject 3 is captured by X-rays generated by radiation generator 2. During practical image capturing, it is required that subject 3 is subjected to image capturing in such a manner that subject 3 is compressed by compressing plate 4 on image capturing stage 7 equipped with radiation image detecting medium (radiation image conversion panel 5) such as a stimulable phosphor. X-rays generated by radiation generator 2 spread radially and reach subject 3, however, in order to minimize adverse effects to a human body and image degradation, the breast wall side portion (being a large shaded area in FIG. 2) is commonly cut.

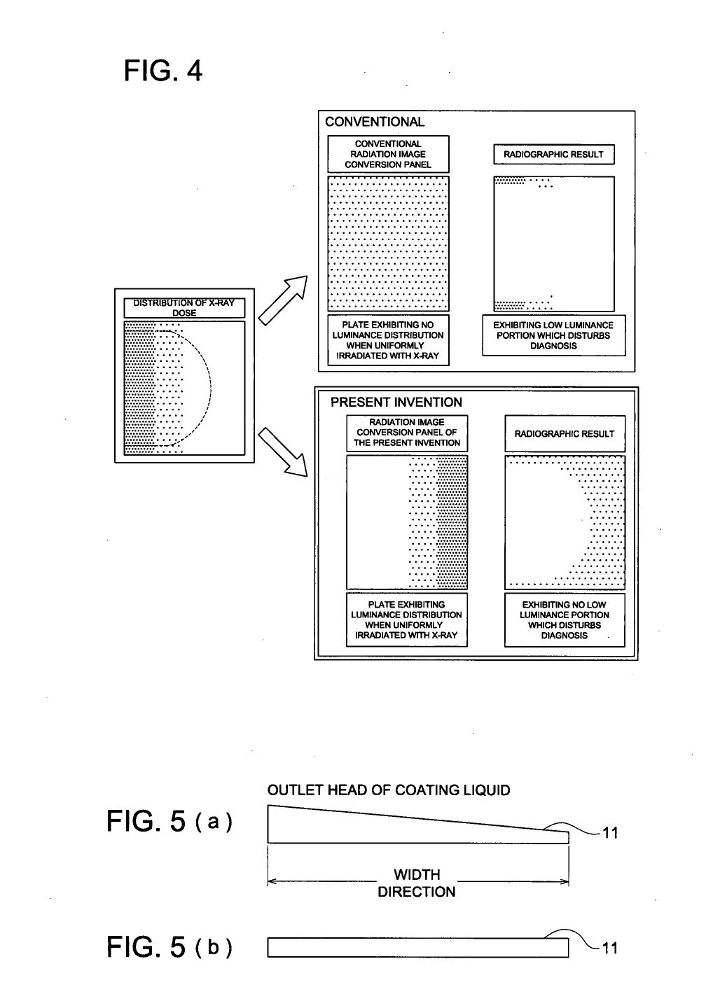

Accordingly, X-ray intensity (being X-ray dose), which reaches subject 3, forms a distribution as shown in FIG. 3 in such a way that the intensity is higher on the near chest wall side and lower on the non-chest wall side. Accordingly, images which have been subjected to the above effects are recorded on radiation image conversion panel 5. In FIG. 4, the above effects and compensated results are shown schematically.

In FIG. 4(a), one schematic view (the drawing on the top side) shows a state in which X-rays having a distribution of the X-ray dose as shown in FIG. 3 is irradiated to subject 3, while the other schematic view (the drawing at the bottom) shows luminance distribution which is obtained in such a manner that after performing recording on the radiation image conversion panel exhibiting uniform responsiveness, the resulting panel results in luminescence.

FIG. 4(b) shows results obtained by practical X-ray exposure. In cases of employing a conventional radiation image conversion panel as shown at the top of FIG. 4(b), no distributed luminance is noted on the radiating image conversion panel. In this case, a final X-ray image exhibits high density on the near chest wall side due to high X-ray dose, and low density on the non-chest wall side.

However, as disclosed in the present invention, when a radiation image conversion panel exhibits distributed luminance, and also when the aforesaid conversion panel is designed to exhibit higher luminance on the non-chest wall side, a uniform image-over the whole area is obtained as shown at the bottom in FIG. 4(b), whereby more accurate diagnosis is possible.

### (Radiation Image Detecting Media (Radiation Image Conversion Panels))

Radiation image detecting media, preferably employed in the present invention, are radiation image conversion panels, which commonly incorporate a support, having thereon a photostimulable phosphor layer and a protective layer. These radiation image conversion panels are sealed with a sealing adhesive or a spacer and sealing adhesive positioned between the support and the protective layer to surround the periphery of the photostimulable phosphor.

Radiation image conversion panels of the present invention exhibits distinct difference between emission luminance on the near chest wall side and on the non-chest wall side. Several production methods of the same may be employed, however such methods are not specifically limited.

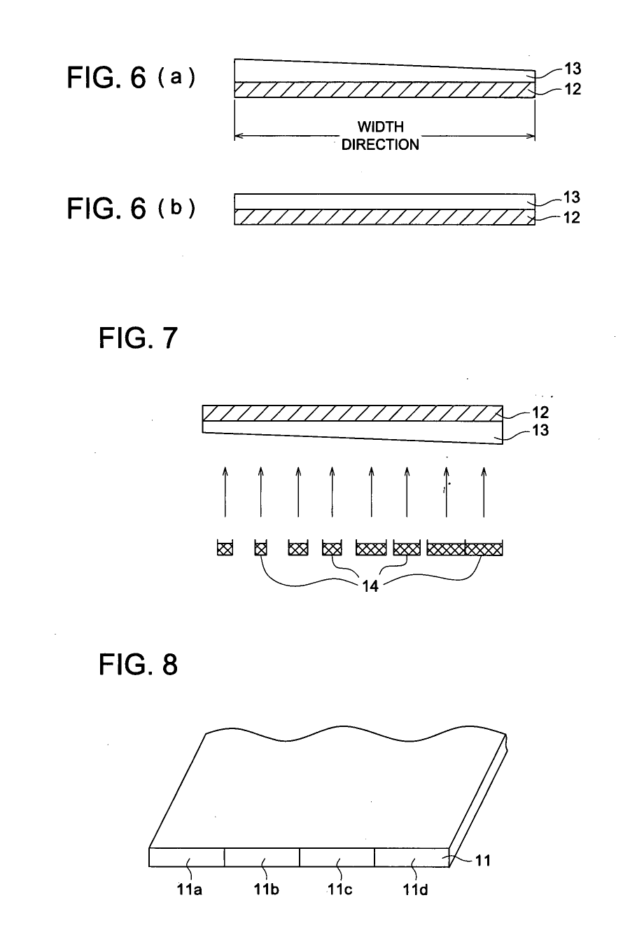

Initially as a typical method, a method is listed in which the thickness of a photostimulable phosphor layer varies. To realize the aforesaid method, the following procedures are carried out: In order to vary the coating weight in the lateral direction, when a photostimulable phosphor layer is formed onto a support using a slide-hopper, the shape of coating mixture outlet 11 varies, that is, the size of the orifice section is changed in the width direction as shown in FIG. 5(a) (the size of the aperture is commonly constant in the width direction as shown in FIG. 5(b)). According to the foregoing, as shown in FIG. 6(a), it is possible to vary the layer thickness of photostimulable phosphor layer 13, which is applied onto support 12, in the lateral direction (layer thickness is commonly constant in the width direction, as shown in FIG. 6(b)).

Further, in cases of production using a vacuum evaporation method, it is possible to vary the deposited amount per unit area on a support by means of gradually enlarging the surface area of evaporation source 14 so as to gradually increase the layer thickness in the width direction as shown in FIG. 7. As a matter of course, changing the distance between the evaporation source and the support may also vary the thickness of a photostimulable phosphor layer.

Further, a method of distributing photostimulable phosphors of differing emission luminance may also be employed in order to distribute emission luminance of a radiation image conversion panel. For this purpose, in cases of employing a slide-hopper coating method, a coating mixture of photostimulable phosphors is coated by dividing coating mixture outlet 11 into several portions as 11a, 11b, 11c, and 11d shown in FIG. 8 so that emission luminance gradually varies over the radiation image conversion panel. Alternatively, in cases of employing a vacuum evaporation method, it is possible to prepare a radiation image conversion panel exhibiting distribution of emission luminance via formation of a photostimulable phosphor layer by means of aligning evaporation source 14 so that emission luminance of the photostimulable phosphor gradually varies when a layer thereof is formed.

Photostimulable phosphors employed in a coating method, for radiation image detecting media of the present invention, include the following: The photostimulable phosphors represented by formula aBaX₂·(1−a)BaY₂:bEu²⁺, described in JP-A No. 2-58593 (wherein X and Y each represent at least one kind of fluorine, chlorine, bromine, or iodine; X is not equal to Y; and a and b each represent any number satisfying 0<a<1 and 10⁻⁵<b<10⁻¹); the alkali halide photostimulable phosphors represented by formula MIX·aMIIX′₂·b MIIIX″₃:cA, described in JP-A No. 61-72087 (wherein MI represents at least one of the alkali metals selected from Li, Na, K, Rb, or Cs; MII represents at least one of the divalent metals selected from Be, Mg, Ca, Sr, Ba, Zn, Cd, Cu, and Ni; MIII represents at least one of the trivalent metals selected from Sc, Y. La, Ce, Pr, Nd, Pm, Sm, Eu, Gd, Tb, Dy, Ho, Er, Tm, Yb, Lu, Al, Ga, or In; X, X′, and X″ represent at least one of halogens selected from fluorine, chlorine, bromine, or iodine; A represents at least one of the metals selected from Eu, Tb, Ce, Tm, Dy, Pr, Ho, Nd, Yb, Er, Gd, Lu, Sm, Y, Tl, Na, Ag, Cu, or Mg; and a, b, and c each represent any number satisfying 0≦a<0.5, 0≦b<0.5, and 0≦c<0.2); photostimulable phosphors represented by formula (Ba₁₋ₓ(MI)ₓFX:yA, described in JP-A No. 55-12145 (wherein MI represents at least one kind of Mg, Ca, Sr, Zn, and Cd; X represents at least one kind of chlorine, bromine, and iodine; A represents at least one kind of Eu, Tb, Ce, Tm, Dy, Pr, Ho, Nd, Yb, and Er; and x and y each represent any number satisfying 0≦x<0.6 and 0≦y<0.2); and the photostimulable phosphors represented by formula MIFX·xA: yLn, described in JP-A No. 55-160078 (wherein MI represents at least one kind of Mg, Ca, Sr, Zn, and Cd; A represents at least one of BeO, MgO, CaO, SrO, BaO, ZnO, Al₂O₃, Y₂O₃, La₂O₃, In₂O₃, Sio₂, TiO₂, ZrO₂, GeO₂, SnO₂, Nb₂O₅, Ta₂O₅, or ThO₂); Ln represents at least one kind of Eu, Tb, Ce, Tm, Dy, Pr, Ho, Nd, Yb, Er, Sm, and Gd; X represents at least one kind of chlorine, bromine, and iodine; and x and y represent any numbers satisfying 5×10⁻⁵≦x≦0.5 and 0<y≦0.2).

Photostimulable phosphors employed in the vacuum evaporation method include the photostimulable phosphors represented by formula M1X·aM2X′₂·bM3X″₃:eA, described in JP-A No. 2004-205354 (wherein M1 represents at least one alkali metal selected from the group containing Li, Na, K, Rb, and Cs; M2 represents at least one divalent metal selected from the group containing Be, Mg, Ca, Sr, Ba, Zn, Cd, Cu, and Ni; M3 represents one trivalent metal selected from the group containing Sc, Y, La, Ce, Pr, Nd, Pm, Sm, Eu, Gd, Tb, Dy, Ho, Er, Tm, Yb, Lu, Al, Ga, and In; X, X′, and X″ represent at least one kind of halogen selected from the group containing fluorine, chlorine, bromine, and iodine; A represents at least one metal selected from the group containing Eu, Tb, In, Ga, Cs, Ce, Tm, Dy, Pr, Ho, Nd, Yb, Er, Gd, Lu, Sm, Y, Tl, Na, Ag, Cu, and Mg; and a, b, and e each represent any number in the range of 0≦a<0.5, 0≦b<0.5, and 0<e≦0.2).

It is preferable that the difference in the above emission luminance over the same radiation image conversion panel is in the range of 5-50% in order to exhibit the effects of the present invention.

### EXAMPLES

The present invention will now be specifically described by referring to the radiation image capturing system for mammography structured as shown in FIG. 2. However, embodiments of the present invention are not limited thereto.

### (Preparation of Radiation Image Conversion Panels 1-6 (Coating Method))

Photostimulable phosphor panels having a cross-section as shown in FIGS. 6(a) and 6(b) were prepared by applying a coating mixture of photostimulable phosphor CsBr:Eu onto a support, employing a slide-hopper with coating mixture outlet 11 having the shape shown in FIGS. 5 (a) and 5(b), followed by drying each of the resulting products. Subsequently, sealed Radiation Image Conversion Panels 1 and 2, each having a protective layer, were prepared by positioning a sealing adhesive between the support and the protective layer at the periphery of the photostimulable phosphor.

Further, photostimulable phosphor panels, each of which has the cross-section shown in FIGS. 6 (a) and 6(b), were prepared by applying a coating mixture of photostimulable phosphor BaFI:Eu onto a support, employing a slide-hopper with coating mixture outlet 11 in the form shown in FIGS. 5 (a) and 5(b), followed by drying each of the resulting products. Subsequently, sealed Radiation Image Conversion Panels 3 and 4, each having a protective layer, were prepared by positioning a sealing adhesive between the support and the protective layer at the periphery of the photostimulable phosphors.

Further, the following coating mixtures were applied onto a support employing a slide hopper having coating mixture outlets shown in FIG. 8. A coating mixture of photostimulable phosphor (CsBr:1.0Eu) with high emission luminance from coating mixture outlet 11a; a coating mixture of photostimulable phosphor (CsBr:0.8Eu), exhibiting low emission luminance from coating mixture outlet lid; a coating mixture obtained by mixing 0.65 part of a mixture of the photostimulable phosphor exhibiting high emission luminance and 0.35 part of a mixture of photostimulable phosphor (CsBr:0.8Eu) exhibiting low emission luminance from coating mixture outlet 11b; and a coating mixture obtained by mixing 0.35 part of a mixture of the photostimulable phosphor exhibiting high emission luminance and 0.65 part of a mixture of the photostimulable phosphor (CsBr:0.8Eu) exhibiting low emission luminance from coating mixture outlet 11c, wherein the discharged amounts of the above coating mixtures were equal. By using the resulting products, sealed Radiation Image Conversion Panel 5, having a protective layer, was prepared by positioning a sealing adhesive between the support and the protective layer at the periphery of the photostimulable phosphor.

Further, the following coating mixtures were applied onto a support employing a slide-hopper with coating mixture outlets shown in FIG. 8: A coating mixture of the photostimulable phosphor (BaFI:1.0Eu) exhibiting high emission luminance from coating mixture outlet 11a; a coating mixture of the photostimulable phosphor (BaFI:0.8Eu) exhibiting low emission luminance from coating mixture outlet lid; a coating mixture obtained by mixing 0.65 part of a mixture of the photostimulable phosphor exhibiting high emission luminance and 0.35 part of a mixture of photostimulable phosphor (CsBr:0.8Eu) exhibiting low emission luminance from coating mixture outlet 11b; and a coating mixture obtained by mixing 0.35 part of the photostimulable phosphor with high emission luminance and 0.65 part of a mixture of photostimulable phosphor (CsBr 0.8Eu) with low emission luminance from coating mixture outlet 11c, wherein the discharged amounts of the above coating mixtures were equal. By using the resulting product, sealed Radiation Image Conversion Panel 6, having a protective layer, was prepared by positioning a sealing adhesive between the support and the protective layer to surround the periphery of the photostimulable phosphor.

### (Preparation of Radiation Image Conversion Panels 7 and 8 (Vacuum Evaporation Method))

Radiation Image Conversion Panel 7 was prepared in the same manner as Radiation Image Conversion Panel 1 except that photostimulable phosphor (CsBr:Eu) was vacuum evaporated by varying the surface area of the evaporation source as shown in FIG. 7.

Radiation Image Conversion Panel 8 was prepared in the same manner as Radiation Image Conversion Panel 7, except that, as commonly conducted, the surface area of the evaporation source is uniform.

### (Radiation Image Capturing for Mammography)

An X-ray image was formed employing a radiation image capturing system for mammography structured shown in FIG. 2 at a tube voltage of 30 kV of a radiation generator, and the image density of the resulting X-ray image at chest wall side was compared to the image density of the resulting X-ray image at non-chest wall side.

Radiation Image Conversion Panels 1, 3, 5, 6, and 7 in the scope of the present invention exhibited uniform emission luminance in the non-image portion, wherein the difference in emission luminance between the maximum and minimum emission luminance was within 0.2, when the maximum emission luminance was set to 1. On the other hand, Radiation Image Conversion Panels 2, 4, and 8, being out of the scope of the present invention, exhibited non-uniform emission luminance in the non-image portion, wherein any difference in emission luminance between the maximum and minimum emission luminance was at least 0.2 when the maximum emission luminance was set to 1, whereby it was possible to obtain only X-ray images in which it was not possible to clearly identify the breast outline at the non-chest wall side of the non-image portion.

### INDUSTRIAL APPLICABILITY

According to the present invention, it is possible to provide a radiation image capturing system for mammography capable of forming high density images suitable for diagnosis even if X-ray dose decreases toward the opposite chest wall from the near chest wall, and a radiation image conversion panel which is suitable for application to the same.

1. A radiation image capturing system for mammography comprising:
a radiation generator exposing a subject to a radiation;
an image capturing stage;
a compressing plate compressing the subject; and
a radiation image conversion panel recording image information of the radiation transmitted through the subject, wherein
an emission luminance of the radiation image conversion panel on a side opposite to a chest wall side is higher than an emission luminance on the chest wall side.

2. The radiation image conversion panel used in the radiation image capturing system for mammography of claim 1, the radiation image conversion panel having the emission luminance of the radiation image conversion panel on the side opposite to the chest wall side higher than the emission luminance on the chest wall side.

3. The radiation image capturing system for mammography of claim 1, wherein
a difference in emission luminance between a maximum emission luminance and a minimum emission luminance in a non-image portion of the radiation image conversion panel after capturing an image is 0.2 or less, provided that the maximum emission luminance is set to 1.

4. The radiation image conversion panel of claim 2, wherein
a difference in emission luminance between a maximum emission luminance and a minimum emission luminance in a non-image portion of the radiation image conversion panel after capturing an image is 0.2 or less, provided that the maximum emission luminance is set to 1.
